(19) United States
(12) Patent Application Publication
(10) Pub. No.: US 2014/0094662 A1
(43) Pub. Date: Apr. 3, 2014

(54) METHOD OF ASSESSING CAPSULAR FORMATION AND/OR CONTRACTURE

(71) Applicant: Allergan, Inc., Irvine, CA (US)

(72) Inventors: Dennis Van Epps, Goleta, CA (US); David Schuessler, Ventura, CA (US); Hilton Kaplan, Los Angeles, CA (US); Rui Avelar, Santa Barbara, CA (US)

(73) Assignee: Allergan, Inc., Irvine, CA (US)

(21) Appl. No.: 14/045,381

(22) Filed: Oct. 3, 2013

Related U.S. Application Data
(60) Provisional application No. 61/709,261, filed on Oct. 3, 2012.

(51) Int. Cl. A61B 5/00

(52) U.S. Cl. CPC A61B 5/48
USPC 600/301

(57) ABSTRACT

The present specification discloses methods of assessing the extent of capsular contracture around an implantable device and methods of monitoring fibrous capsular formation relating to an implantable device.

This application claims the benefit of, and priority to, U.S. Provisional Patent Application No. 61/709,261, filed Oct. 3, 2012, the entire disclosure of this is incorporated herein by this reference.

Implantation of a device in an individual involves interaction of the device with host tissue in the individual and generally results in the formation of a soft-tissue fibrous capsule (or encapsulation) around the implant, which is a normal process and is related to the scarification process. In some cases capsular contracture (also known as capsular contraction) may occur, which is the thickening, tightening, and eventual hardening of the fibrous capsule that surrounds the implant. This capsular contracture in some implants provides positive effects, such as holding the device (e.g., knee or hip implant) in place. In other instances the contracture can produce negative effects, such as, causing visible distortions of the shape of the tissue surrounding the implant. In the case of breast implants, contracture results in symptoms such as breast firmness, pain, and visible distortions of the shape of the breast.

As such, there is a need for methods for accurately assessing an occurrence and/or extent of capsular formation and/or an occurrence and/or extent of capsular contraction after implant of a device. Such assessment can provide clinically relevant information including, without limitation, the degree and/or progress of healing after implantation of a device, the location and/or placement of an implanted device, the degree and/or progress of proper integration of an implanted device in the body, the presence or absence of capsular formation and/or capsular contraction, the degree and progress of capsular formation and/or capsular contraction, the necessity of surgical removal or release of the capsule, or the necessity of surgical removal and/or possible replacement of the device itself.

The methods disclosed herein assess an occurrence and/or extent of capsular formation and/or capsular contracture around an implanted device in an individual. This is accomplished by one or more qualitative and/or quantitative assessments of an area in the vicinity of the implanted device.

### SUMMARY

Thus, aspects of the present specification disclose methods for assessing occurrence and/or extent of capsular formation and/or occurrence and/or extent of capsular contraction in and around an implanted device in an individual. The disclosed methods comprise obtaining one or more assessments of an area in the vicinity of the implanted device. Assessments may be quantitative, qualitative or a combination of both. Assessments include, without limitation, depth measurements, tissue integration measurements, tonometry measurements, physical examination assessments, anatomical assessments, photographic assessments, or any combination thereof.

Other aspects of the present specification disclose methods for assessing occurrence and/or extent of capsular formation and/or occurrence and/or extent of capsular contraction in and around a breast implant in an individual. The disclosed methods comprise obtaining one or more assessments of an area in the vicinity of the implanted device. Assessments may be quantitative measurements, qualitative measurements, or a combination of both. Assessments include, without limitation, depth measurements, tissue integration measurements, tonometry measurements, physical examination assessments, anatomical assessments, photographic assessments, or any combination thereof.

Yet other aspects of the present specification disclose methods for monitoring capsular formation and/or contracture formation in and around an implanted device in an individual. The disclosed methods comprise (a) obtaining one or more assessments of an area in the vicinity of the implanted device at a first time point; (b) obtaining one or more assessments of the area in the vicinity of the implanted device at one or more subsequent time points; and, (c) comparing the one or more assessments obtained in step (a) with the one or more assessments obtained in step (b) to assess occurrence and/or extent of capsular formation and/or occurrence and/or extent of capsular contraction in and around the implanted device, thereby monitoring capsular formation and contracture in and around the implanted device. Assessments may be quantitative measurements, qualitative measurements, or a combination of both. Assessments include, without limitation, depth measurements, tissue integration measurements, tonometry measurements, physical examination assessments, anatomical assessments, photographic assessments, or any combination thereof.

### BRIEF DESCRIPTION OF THE DRAWINGS

FIG. 1 shows a cross-sectional view of a subglandular breast implant.

FIG. 2 shows a cross-sectional view of a submuscular breast implant.

### DETAILED DESCRIPTION

Aspects of the present specification provide an implanted device. As used herein, the term “implanted device” refers to any biocompatible implantable device. As used herein, the term “implanted” refers to the embedding of the device into, or attachment of the device to, tissue, muscle, organ or any other part of an animal body. As used herein, the term “individual” relates to an animal and includes all mammals, including a human. An “implanted device” is synonymous with “biocompatible implantable device,” “medical device,” “biomedical device,” “implantable medical device,” “implantable biomedical device,” etc. and includes, without limitation, pacemakers, dura matter substitutes, implantable cardiac defibrillators, tissue expanders, and tissue implants used for prosthetic, reconstructive, or aesthetic purposes, like breast implants, muscle implants, or implants that reduce or prevent scarring.

Some examples of implanted devices for which the disclosed methods may be useful to assess occurrence and/or extend of capsular contraction and/or occurrence and/or extent of capsular contraction are described in, e.g., Schuessler and Powell, All-Barrier Elastomer Gel-Filled Breast Prosthesis, U.S. Pat. No. 8,043,373; Schuessler, Rotational Molding System for Medical Articles, U.S. Pat. No. 7,628,604; Smith, Mastopexy Stabilization Apparatus and Method, U.S. Pat. No. 7,081,135; Knisley, Inflatable Prosthetic Device, U.S. Pat. No. 6,936,068; Falcon, Reinforced Radius Mammary Prostheses and Soft Tissue Expanders, U.S. Pat. No. 6,605,116; Schuessler, Rotational Molding of Medical Articles, U.S. Pat. No. 6,602,452; Murphy, Seamless Breast Prosthesis, U.S. Pat. No. 6,074,421; Knowlton, Segmental Breast Expander For Use in Breast Reconstruction, U.S. Pat. No. 6,071,309; VanBeek, Mechanical Tissue Expander, U.S. Pat. No. 5,882,353; Hunter, Soft Tissue Implants and Anti-Scarring Agents, U.S. 2010/0049317; Schuessler, Self-Sealing Shell For Inflatable Prostheses, U.S. 2009/0214652; Schraga, Medical Implant Containing Detection Enhancing Agent and Method For Detecting Content Leakage, U.S. 2009/0157180; Connell, Differential Tissue Expander Implant, U.S. 2007/0233273; and Hunter, Medical implants and Anti-Scarring Agents, U.S. 2006/0147492; Van Epps, Soft Filled Prosthesis Shell with Discrete Fixation Surfaces, WO 2010/019761; Schuessler, Self Sealing Shell for Inflatable Prosthesis, WO 2010/022130; Yacoub, Prosthesis Implant Shell, International Application No. PCT/US09/61045; Liu, et al., Porous Materials, Methods of making and Uses, U.S. 2011/0282444; Liu, et al., Porous Materials, Methods of making and Uses, U.S. patent application Ser. No. 13/625,159; Liu, et al., Porogen Compositions, Methods of Making and Uses, U.S. 2011/0278755, Liu, et al., Porogen Compositions, Methods of Making and Uses, U.S. patent application Ser. No. 13/631,091, each of which is hereby incorporated by reference in its entirety.

An implanted device disclosed herein can be implanted into the soft tissue of an individual during the normal operation of the device. Such implanted devices may be completely implanted into the soft tissue of an animal body (i.e., the entire device is implanted within the body), or the device may be partially implanted into an animal body (i.e., only part of the device is implanted within an animal body, the remainder of the device being located outside of the animal body).

Implantable medical devices frequently induce a foreign body response that results in the formation of an avascular, fibrous capsule around the implant. As used herein, “capsular formation” refers to the formation of this fibrous capsule around an implanted device in an individual. The “fibrous capsule” is synonymous with “tissue capsule,” “scar capsule,” and “fibrous soft tissue capsule” and like terms, and may be referred to herein simply as “capsule.” The fibrous capsule is a lining of soft tissue including fibroblasts and collagen fibers that generally surrounds the implanted device, and is a natural host response to a foreign object placed within the body.

The present specification discloses, in part, a porous material that covers a surface of the biocompatible implantable device. Any of the porous materials disclosed herein can be used as the porous material covering a surface of a biocompatible implantable device. In general, the surface of a biocompatible implantable device is one exposed to the surrounding tissue of an animal in a manner that promotes tissue growth, and/or reduces or prevents formation of fibrous capsules that can result in capsular contracture or scarring.

A biocompatible implantable device may be a base shell comprising a single layer or a plurality of layers. In an aspect of this embodiment, a base shell comprises one or more inner base layer of a substance or elastomer, a barrier or reinforcement layer and one or more outer base layer of a substance or an elastomer, wherein the barrier or reinforcement layer lays in between the one or more inner base layers and one or more outer base layers. In another aspect of this embodiment, a base shell comprises one inner base layer of a substance or an elastomer, a barrier or reinforcement layer and two outer base layer of a substance or an elastomer. In yet another aspect of this embodiment, a base shell comprises two inner base layers of a substance or an elastomer, a barrier or reinforcement layer and two outer base layers of a substance or an elastomer. In still another aspect of this embodiment, a base shell comprises two inner base layers of a substance or an elastomer, a barrier or reinforcement layer and three outer base layers of a substance or an elastomer. The barrier or reinforcement layer may comprise a synthetic polymer mesh or fabric. Exemplary base shells include, without limitation, a breast implant shell or a tissue expander shell.

Thus, in an embodiment, a porous material covers the entire surface of a biocompatible implantable device. In another embodiment, a porous material covers a portion of a surface of a biocompatible implantable device. In aspects of this embodiment, a porous material covers to a front surface of a biocompatible implantable device or a back surface of a biocompatible implantable device. In other aspects, a porous material covers only to, e.g., about 20%, about 30%, about 40%, about 50%, about 60%, about 70% about 80% or about 90% of the entire surface of a biocompatible implantable device, a front surface of a biocompatible implantable device, or a back surface of a biocompatible implantable device. In yet other aspects, a porous material is applied only to, e.g., at least 20%, at least 30%, at least 40%, at least 50%, at least 60%, at least 70% at least 80% or at least 90% of the entire surface of a biocompatible implantable device, a front surface of a biocompatible implantable device, or a back surface of a biocompatible implantable device. In still other aspects, a porous material is applied only to, e.g., at most 20%, at most 30%, at most 40%, at most 50%, at most 60%, at most 70% at most 80% or at most 90% of the entire surface of a biocompatible implantable device, a front surface of a biocompatible implantable device, or a back surface of a biocompatible implantable device. In further aspects, a porous material is applied only to, e.g., about 20% to about 100%, about 30% to about 100%, about 40% to about 100%, about 50% to about 100%, about 60% to about 100%, about 70% to about 100%, about 80% to about 100%, or about 90% to about 100% of the entire surface of a biocompatible implantable device, a front surface of a biocompatible implantable device, or a back surface of a biocompatible implantable device.

The layer of porous material covering a biocompatible implantable device can be of any thickness with the proviso that the material thickness allows tissue growth within the array of interconnected of pores of a substance matrix in a manner sufficient to reduce or prevent formation of fibrous capsules that can result in capsular contracture or scarring.

In an embodiment, a layer of porous material covering a biocompatible implantable device is of a thickness that allows tissue growth within the array of interconnected of pores of a substance matrix in a manner sufficient to reduce or prevent formation of fibrous capsules that can result in capsular contracture or scarring. In aspects of this embodiment, a layer porous material covering a biocompatible implantable device comprises a thickness of, e.g., about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm. In other aspects of this embodiment, a layer porous material covering a biocompatible implantable device comprises a thickness of, e.g., at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, at least 5 mm, at least 6 mm, at least 7 mm, at least 8 mm, at least 9 mm, or at least 10 mm. In yet other aspects of this embodiment, a layer porous material covering a biocompatible implantable device comprises a thickness of, e.g., at most 100 μm, at most 200 μm, at most 300 μm, at most 400 μm, at most 500 μm, at most 600 μm, at most 700 μm, at most 800 μm, at most 900 μm, at most 1 mm, at most 2 mm, at most 3 mm, at most 4 mm, at most 5 mm, at most 6 mm, at most 7 mm, at most 8 mm, at most 9 mm, or at most 10 mm. In still other aspects of this embodiment, a layer porous material covering a biocompatible implantable device comprises a thickness of, e.g., about 100 μm to about 500 μm, about 100 μm to about 1 mm, about 100 μm to about 5 mm, about 300 μm to about 1 mm, about 300 μm to about 2 mm, about 300 μm to about 3 mm, about 300 μm to about 4 mm, about 300 μm to about 5 mm, about 500 μm to about 1 mm, about 500 μm to about 2 mm, about 500 μm to about 3 mm, about 500 μm to about 4 mm, about 500 μm to about 5 mm, about 800 μm to about 1 mm, about 800 μm to about 2 mm, about 800 μm to about 3 mm, about 800 μm to about 4 mm, about 800 μm to about 5 mm, about 1 mm to about 2 mm, about 1 mm to about 3 mm, about 1 mm to about 4 mm, about 1 mm to about 5 mm, or about 1.5 mm to about 3.5 mm.

In an embodiment, a layer of porous material disclosed herein may comprises a substance matrix defining an array of interconnected pores has a porosity sufficient to allow tissue growth into the array of interconnected pores. In aspects of this embodiment, a porous material comprising a substance matrix comprises a porosity of, e.g., about 40% of the total volume of a substance matrix, about 50% of the total volume of a substance matrix, about 60% of the total volume of a substance matrix, about 70% of the total volume of a substance matrix, about 80% of the total volume of a substance matrix, about 90% of the total volume of a substance matrix, about 95% of the total volume of a substance matrix, or about 97% of the total volume of a substance matrix. In other aspects of this embodiment, a porous material comprising a substance matrix comprises a porosity of, e.g., at least 40% of the total volume of a substance matrix, at least 50% of the total volume of a substance matrix, at least 60% of the total volume of a substance matrix, at least 70% of the total volume of a substance matrix, at least 80% of the total volume of a substance matrix, at least 90% of the total volume of a substance matrix, at least 95% of the total volume of a substance matrix, or at least 97% of the total volume of a substance matrix. In yet other aspects of this embodiment, a porous material comprising a substance matrix comprises a porosity of, e.g., at most 40% of the total volume of a substance matrix, at most 50% of the total volume of a substance matrix, at most 60% of the total volume of a substance matrix, at most 70% of the total volume of a substance matrix, at most 80% of the total volume of a substance matrix, at most 90% of the total volume of a substance matrix, at most 95% of the total volume of a substance matrix, or at most 97% of the total volume of a substance matrix. In yet other aspects of this embodiment, a porous material comprising a substance matrix comprises a porosity of, e.g., about 40% to about 97% of the total volume of a substance matrix, about 50% to about 97% of the total volume of a substance matrix, about 60% to about 97% of the total volume of a substance matrix, about 70% to about 97% of the total volume of a substance matrix, about 80% to about 97% of the total volume of a substance matrix, about 90% to about 97% of the total volume of a substance matrix, about 40% to about 95% of the total volume of a substance matrix, about 50% to about 95% of the total volume of a substance matrix, about 60% to about 95% of the total volume of a substance matrix, about 70% to about 95% of the total volume of a substance matrix, about 80% to about 95% of the total volume of a substance matrix, about 90% to about 95% of the total volume of a substance matrix, about 40% to about 90% of the total volume of a substance matrix, about 50% to about 90% of the total volume of a substance matrix, about 60% to about 90% of the total volume of a substance matrix, about 70% to about 90% of the total volume of a substance matrix, or about 80% to about 90% of the total volume of a substance matrix.

In another embodiment, a porous material disclosed herein may comprise a substance matrix includes a surface openness sufficient to allow tissue growth into the array of interconnected pores. Surface openness, or first level openness, refers to the percentage area that the pores at the surface of a porous material are exposed to the surroundings. Surface openness may be determined by examining a top view image of a porous material. In aspects of this embodiment, a porous material comprising a substance matrix includes a surface openness of, e.g., about 45%, about 50%, about 55%, about 60%, about 65%, about 70%, about 75%, about 80%, about 85%, about 90%, about 93%, about 95%, about 97%, or about 100%. In other aspects of this embodiment, a porous material comprising a substance matrix includes a surface openness of, e.g., at least 45%, at least 50%, at least 55%, at least 60%, at least 65%, at least 70%, at least 75%, at least 80%, at least 85%, at least 90%, at least 93%, at least 95%, at least 97%, or at least 100%. In yet other aspects of this embodiment, a porous material comprising a substance matrix includes a surface openness of, e.g., about 45% to about 100%, about 50% to about 100%, about 55% to about 100%, about 60% to about 100%, about 65% to about 100%, about 70% to about 100%, about 75% to about 100%, about 80% to about 100%, or about 85% to about 100%.

In another embodiment, a porous material disclosed herein may comprise a substance matrix includes an interconnectivity between pores sufficient to allow tissue growth into the array of interconnected pores. Interconnectivity, or second level openness, may be determined by measuring the area of visible openings or interconnections within each pore or surface opening from a top view image of a porous material and relating that area to the total area of the analyzed image. In aspects of this embodiment, a porous material comprising a substance matrix includes an interconnectivity between pores of, e.g., about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90%, about 93%, or about 95%. In other aspects of this embodiment, a porous material comprising a substance matrix includes an interconnectivity between pores of, e.g., at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 93%, or at least 95%. In yet other aspects of this embodiment, a porous material comprising a substance matrix includes an interconnectivity between pores of, e.g., about 8% to about 20%, about 9% to about 20%, about 10% to about 20%, about 11% to about 20%, about 12% to about 20%, about 13% to about 20%, about 14% to about 20%, or about 15% to about 20%. In yet other aspects of this embodiment, a porous material comprising a substance matrix includes an interconnectivity between pores of, e.g., about 6% to about 22%, about 7% to about 21%, about 8% to about 20%, about 9% to about 19%, about 10% to about 18%, about 11% to about 17%, about 12% to about 16%, about 13% to about 15%, about 20% to about 60%, about 25% to about 60%, about 30% to about 60%, about 35% to about 60%, about 40% to about 60%, about 45% to about 60%, about 50% to about 60%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 20% to about 95%, about 25% to about 95%, about 30% to about 95%, about 35% to about 95%, about 40% to about 95%, about 45% to about 95%, about 50% to about 95%, about 55% to about 95%, about 60% to about 95%, about 65% to about 95%, about 70% to about 95%, about 75% to about 95%, about 80% to about 95%, or about 85% to about 95%.

In another embodiment, a porous material disclosed herein may comprise a substance matrix includes a thickness to allow tissue growth into the array of interconnected pores. For example, a porous material may be from about 0.1 mm to about 1 mm, about 0.25 mm to about 1.5 mm, about 0.25 mm to about 2.5 mm, or about 0.5 mm to about 5 mm in thickness. In aspects of this embodiment, a porous material comprises a thickness of, e.g., about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 10 mm. In other aspects of this embodiment, a porous material comprises a thickness of, e.g., at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, at least 5 mm, at least 6 mm, at least 7 mm, at least 8 mm, at least 9 mm, or at least 10 mm. In yet other aspects of this embodiment, a porous material comprises a thickness of, e.g., at most 100 μm, at most 200 μm, at most 300 μm, at most 400 μm, at most 500 μm, at most 600 μm, at most 700 μm, at most 800 μm, at most 900 μm, at most 1 mm, at most 2 mm, at most 3 mm, at most 4 mm, at most 5 mm, at most 6 mm, at most 7 mm, at most 8 mm, at most 9 mm, or at most 10 mm. In still other aspects of this embodiment, a porous material comprises a thickness of, e.g., about 100 μm to about 500 μm, about 100 μm to about 1 mm, about 100 μm to about 5 mm, about 300 μm to about 1 mm, about 300 μm to about 2 mm, about 300 μm to about 3 mm, about 300 μm to about 4 mm, about 300 μm to about 5 mm, about 500 μm to about 1 mm, about 500 μm to about 2 mm, about 500 μm to about 3 mm, about 500 μm to about 4 mm, about 500 μm to about 5 mm, about 800 μm to about 1 mm, about 800 μm to about 2 mm, about 800 μm to about 3 mm, about 800 μm to about 4 mm, about 800 μm to about 5 mm, about 1 mm to about 2 mm, about 1 mm to about 3 mm, about 1 mm to about 4 mm, about 1 mm to about 5 mm, or about 1.5 mm to about 3.5 mm.

In another embodiment, a porous material disclosed herein may comprise a substance matrix includes substantially no trapped porogens within the cured elastomer matrix. Porogens may become trapped within the cured substance matrix in situations where there is no interconnection with other pores. In aspects of this embodiment, a porous material comprising a substance matrix comprises, e.g., about 1 porogens/mg of porous material, about 2 porogens/mg of porous material, about 4 porogens/mg of porous material, about 5 porogens/mg of porous material, about 6 porogens/mg of porous material, about 8 porogens/mg of porous material, about 10 porogens/mg of porous material, about 15 porogens/mg of porous material, or about 20 porogens/mg of porous material. In other aspects of this embodiment, a porous material comprising a substance matrix comprises, e.g., at most 1 porogens/mg of porous material, at most 2 porogens/mg of porous material, at most 4 porogens/mg of porous material, at most 5 porogens/mg of porous material, at most 6 porogens/mg of porous material, at most 8 porogens/mg of porous material, at most 10 porogens/mg of porous material, at most 15 porogens/mg of porous material, or at most 20 porogens/mg of porous material. In yet other aspects of this embodiment, a porous material comprising a substance matrix comprises, e.g., about 1 porogens/mg of porous material to about 5 porogens/mg of porous material, about 1 porogens/mg of porous material to about 10 porogens/mg of porous material, about 1 porogens/mg of porous material to about 15 porogens/mg of porous material, or about 1 porogens/mg of porous material to about 20 porogens/mg of porous material.

In aspects of this embodiment, a porous material disclosed herein may comprise a substance matrix comprises, e.g., about 50 porogens, about 100 porogens, about 200 porogens, about 300 porogens, about 400 porogens, about 500 porogens, about 600 porogens, about 700 porogens, about 800 porogens, about 900 porogens, or about 1000 porogens. In other aspects of this embodiment, a porous material comprising a substance matrix comprises, e.g., at most 50 porogens, at most 100 porogens, at most 200 porogens, at most 300 porogens, at most 400 porogens, at most 500 porogens, at most 600 porogens, at most 700 porogens, at most 800 porogens, at most 900 porogens, or at most 1000 porogens. In yet other aspects of this embodiment, a porous material comprising a substance matrix comprises, e.g., about 50 porogens to about 100 porogens, about 50 porogens to about 200 porogens, about 50 porogens to about 300 porogens, about 50 porogens to about 400 porogens, about 50 porogens to about 500 porogens, about 50 porogens to about 600 porogens, about 50 porogens to about 700 porogens, about 50 porogens to about 800 porogens, about 50 porogens to about 900 porogens, about 50 porogens to about 1000 porogens, about 200 porogens to about 300 porogens, about 200 porogens to about 400 porogens, about 200 porogens to about 500 porogens, about 200 porogens to about 600 porogens, about 200 porogens to about 700 porogens, about 200 porogens to about 800 porogens, about 200 porogens to about 900 porogens, about 200 porogens to about 1000 porogens, about 500 porogens to about 600 porogens, about 500 porogens to about 700 porogens, about 500 porogens to about 800 porogens, about 500 porogens to about 900 porogens, or about 500 porogens to about 1000 porogens.

In another embodiment, a porous material disclosed herein may comprise a substance matrix includes pores where the diameter of the connections between pores is sufficient to allow tissue growth into the array of interconnected pores. In aspects of this embodiment, a porous material comprising a substance matrix includes pores where the diameter of the connections between pores is, e.g., about 10% the mean pore diameter, about 20% the mean pore diameter, about 30% the mean pore diameter, about 40% the mean pore diameter, about 50% the mean pore diameter, about 60% the mean pore diameter, about 70% the mean pore diameter, about 80% the mean pore diameter, or about 90% the mean pore diameter. In other aspects of this embodiment, a porous material comprising a substance matrix includes pores where the diameter of the connections between pores is, e.g., at least 10% the mean pore diameter, at least 20% the mean pore diameter, at least 30% the mean pore diameter, at least 40% the mean pore diameter, at least 50% the mean pore diameter, at least 60% the mean pore diameter, at least 70% the mean pore diameter, at least 80% the mean pore diameter, or at least 90% the mean pore diameter. In yet other aspects of this embodiment, a porous material comprising a substance matrix includes pores where the diameter of the connections between pores is, e.g., at most 10% the mean pore diameter, at most 20% the mean pore diameter, at most 30% the mean pore diameter, at most 40% the mean pore diameter, at most 50% the mean pore diameter, at most 60% the mean pore diameter, at most 70% the mean pore diameter, at most 80% the mean pore diameter, or at most 90% the mean pore diameter.

“Capsular contracture,” also known as “capsular contraction,” or “contracture,” refers to tightening and hardening of the fibrous capsule that has formed around the implanted device. Capsular contracture may limit the performance of the device. In some cases capsular contracture can cause the individual discomfort and pain. Especially where the device is implanted in soft tissue, contracture can also dramatically alter the visible shape of the anatomical area surrounding the device, distorting the aesthetic appearance of the area, or make the area unnaturally firm or hard. An example of this is where the implanted device is a breast implant.

Problems with capsular formation and contracture occur in many types of implantable medical devices, such as, e.g., pacemakers, orthopedic joint prosthetics, dura matter substitutes, implantable cardiac defibrillators, tissue expanders, and tissue implants used for prosthetic, reconstructive, or aesthetic purposes, like breast implants, muscle implants, or implants that are meant to reduce or prevent scarring. Capsular contracture is in fact one of the most common complications following breast reconstruction or augmentation. Accordingly, some embodiments of the present disclosure relate to methods of assessing the extent of capsular contracture around a breast implant in an individual.

The topology of the implanted device can be associated with occurrence and/or extent of capsular formation and/or occurrence and/or extent of contracture. The biological response to implanted devices and wound healing appears dependent on the microarchitecture of the surface of the device. Implanted devices with smooth surfaces in particular are most susceptible to capsular formation and contracture. One means of reducing capsular formation and contracture then is to texture the surface of the implanted device, for example by imprinting texture onto the surface of the device, or by overlaying the device with a sheet or film of textured material. The planar and directional arrangement that fibroblasts have been observed to adopt around an implanted device having a smooth outer surface, may induce the formation of synovial epithelium and the secretion of inflammatory factors leading to increased capsule tightening and hardening. In contrast, implanted devices having a textured outer surface have been observed to promote fibroblast in-growth in the implant, integrating the implant with the host tissue. As one example, implants having a textured surface comprising layers of interconnected pores provide a deeper substrate for tissue integration following implantation. Where the outer surface of the implanted device is manufactured such that it possesses thicker texturing and multiple interconnected porous layers, the result is increased tissue integration. The end result is that, in contrast to the body segregating the implant following implantation, a porous surface is provided on the implant which promotes implant-tissue integration and thus reduces capsule formation and/or capsular contracture.

Accordingly, in some embodiments, methods of assessing an occurrence and/or extent capsular formation and/or an occurrence and/or extent capsular contracture relate to an implanted device comprising a smooth outer surface. Other embodiments relate to a device comprising a textured outer surface. In specific embodiments of the present disclosure relating to a device comprising a textured outer surface, the device comprises a flexible elastomer shell, wherein the elastomer shell includes an outer layer of porous material comprising a substantially non-degradable, biocompatible, elastomer matrix defining an array of interconnected pores. In some embodiments the flexible elastomer shell is formed by (a) coating a cured elastomer base layer with an uncured elastomer base, (b) coating the uncured elastomer base with porogens to form an elastomer coated porogen mixture, (c) coating the elastomer coated porogen mixture with an uncured elastomer base, (d) repeating steps (b) and (c), (e) treating the elastomer coated porogen mixture such that the porogens are fused and the elastomer is cured, and (f) removing the porogen scaffold, thus forming the flexible elastomer shell comprising a porous material outer layer comprising more than one layer of interconnected pores. In some embodiments of the present disclosure, the implanted device is a breast implant.

“Elastomer” as used herein can refer to a natural or synthetic elastomer or elastic polymer, and refers to an amorphous polymer that exists above its glass transition temperature at ambient temperatures, thereby conferring the property of viscoelasticity so that considerable segmental motion is possible, and includes, without limitation, carbon-based elastomers, silicon-based elastomers, thermoset elastomers, and thermoplastic elastomers. As used herein, the term “ambient temperature” refers to a temperature of about 18° C. to about 22° C. Elastomers, ether naturally occurring or synthetically made, comprise monomers usually made of carbon, hydrogen, oxygen, and/or silicon which are linked together to form long polymer chains. Elastomers are typically covalently cross-linked to one another, although non-covalently cross-linked elastomers are known. Elastomers may be homopolymers or copolymers, degradable, substantially non-degradable, or non-degradable. Copolymers may be random copolymers, blocked copolymers, graft copolymers, and/or mixtures thereof. Unlike other polymers classes, elastomers can be stretched many times its original length without breaking by reconfiguring themselves to distribute an applied stress, and the cross-linkages ensure that the elastomers will return to their original configuration when the stress is removed. Elastomers can be a non-medical grade elastomer or a medical grade elastomer. Medical grade elastomers are typically divided into three categories: non-implantable, short term implantable and long-term implantable. Exemplary substantially non-degradable and/or non-degradable, biocompatible, elastomers include, without limitation, bromo isobutylene isoprene (BIIR), polybutadiene (BR), chloro isobutylene isoprene (CIIR), polychloroprene (CR), chlorosulphonated polyethylene (CSM), ethylene propylene (EP), ethylene propylene diene monomer (EPDM), fluoronated hydrocarbon (FKM), fluoro silicone (FVQM), hydrogenated nitrile butadiene (HNBR), polyisoprene (IR), isobutylene isoprene butyl (IIR), methyl vinyl silicone (MVQ), acrylonitrile butadiene (NBR), polyurethane (PU), styrene butadiene (SBR), styrene ethylene/butylene styrene (SEBS), polydimethylsiloxane (PDMS), polysiloxane (SI), and acrylonitrile butadiene carboxy monomer (XNBR).

Assessments of an occurrence and/or extent of capsular formation and/or an occurrence and/or extent of capsular contraction according to the present disclosure can be qualitative, quantitative, or a combination thereof. A qualitative assessment is a subjective assessment based on an observation of the patient. A quantitative assessment is an objective assessment based on a measurement of some parameter that can be numerically quantified. Assessing an occurrence and/or extent of capsular formation and/or an occurrence and/or extent of capsular contraction, as understood herein, can include assessing or detecting formation of the fibrous capsule, assessing or measuring the thickness and/or firmness of the capsule, assessing or measuring integration of the implanted device with the surrounding tissue, assessing or detecting formation of capsular contracture, and assessing the relative severity of contracture once formed. As such, methods of assessing the extent of contracture typically include obtaining one or more assessments of the area in the vicinity of the implanted device.

Assessments disclosed herein are obtained from an area in the vicinity of the implanted device. An area in the vicinity of the implanted device includes, without limitation, a surface or internal bodily area of an individual directly overlaying or underlying the implanted device, a surface or internal bodily area of an individual adjacent to the implanted device, a surface or internal bodily area of an individual from about 0.1 cm to about 10 cm from the implanted device, or any other a surface or internal bodily area that can provide statistically relevant or otherwise meaningful information to assess occurrence and/or extent of capsular formation and/or occurrence and/or extent of capsular contraction. In aspects of this embodiment, an area in the vicinity of the implanted device include the areas directly underlying the implanted device, areas nearby, but not directly underlying the implanted device, or any other area affected by implantation of the device.

Assessments of an occurrence and/or extent capsular formation and/or an occurrence and/or extent capsular contracture may include, e.g., a single qualitative assessment, a single quantitative assessment, a plurality of qualitative assessments and a single quantitative assessment, a single qualitative assessment and a plurality of quantitative assessments, or a plurality of qualitative assessment and a plurality of quantitative assessments. In aspects of this embodiment, methods of assessing an occurrence and/or extent capsular formation and/or an occurrence and/or extent capsular contracture around an implanted device comprises obtaining one or more, two or more, three or more, four or more, five or more, six or more or seven or more qualitative assessments. In other aspects of this embodiment, methods of assessing an occurrence and/or extent capsular formation and/or an occurrence and/or extent capsular contracture around an implanted device comprises obtaining one or more, two or more, three or more, four or more, five or more, six or more or seven or more quantitative assessments. In yet other aspects of this embodiment, methods of assessing an occurrence and/or extent capsular formation and/or an occurrence and/or extent capsular contracture around an implanted device comprises obtaining one or more, two or more, three or more, four or more, five or more, six or more or seven or more quantitative assessments and obtaining one or more, two or more, three or more, four or more, five or more, six or more or seven or more quantitative assessments.

In other aspects of this embodiment, methods of assessing an occurrence and/or extent capsular formation and/or an occurrence and/or extent capsular contracture around an implanted device is determined without obtaining a qualitative assessment. In yet other aspects of this embodiment, methods of assessing an occurrence and/or extent capsular formation and/or an occurrence and/or extent capsular contracture around an implanted device comprises obtaining one or more, two or more, three or more, four or more, five or more, six or more or seven or more quantitative assessments, with the proviso that no qualitative assessments are obtained.

Examples of qualitative assessments useful for assessing an occurrence and/or extent capsular formation and/or an occurrence and/or extent capsular contracture may include, without limitation, physical examination assessments, anatomical assessments, photographic assessments, or any combination thereof.

A qualitative assessment by physical examination can include an evaluation of the satisfaction of implant performance by a healthcare provider, an evaluation of the satisfaction of implant performance by the individual with the implant, evaluating whether the implant is palpably distinguishable from surrounding tissue, and/or evaluating the presence of capsular contraction itself, which evaluation can be based on the look and feel of the tissue surrounding the implant. Implant performance generally relates to assessing an occurrence and/or extent capsular formation and/or an occurrence and/or extent capsular contracture. Thus, the higher the satisfaction of implant performance, the less likely there was capsular formation and/or contracture. Conversely, the higher the dissatisfaction of implant performance, the more likely there was capsular formation and/or contracture.

In some embodiments, a physical examination assessment includes evaluating satisfaction of a healthcare provider with implant performance may be measured using a questionnaire. The questionnaire can contain questions relating to, for example but without limitation, healthcare provider satisfaction with the implant itself, satisfaction with the over-all well-being of the individual with the implant after implantation, psychological or psychosocial well-being of the individual with the implant after implantation, and satisfaction with over-all outcome of the implant. In some embodiments, the questionnaire can also contain additional questions specifically tailored to the type of implant, such as, for example in the case of breast augmentation, aesthetic appearance.

Evaluating satisfaction of a healthcare provider with implant performance may be measured using a scale to quantify the subjective assessment of the healthcare provider. In one embodiment, implant performance is measured using a two point scale where 1 indicates the healthcare provider is dissatisfied with implant performance and 2 indicates the healthcare provider is satisfied with implant performance. In another embodiment, implant performance is measured using a three point scale where 1 indicates the healthcare provider is dissatisfied with implant performance, 2 indicates the healthcare provider is neither dissatisfied nor satisfied with implant performance, and 3 indicates the healthcare provider is definitely satisfied with implant performance. In yet another embodiment, implant performance is measured using a four point scale where 1 indicates the healthcare provider is strongly dissatisfied with implant performance, 2 indicates the healthcare provider is somewhat dissatisfied with implant performance, 3 indicates the healthcare provider is somewhat satisfied with implant performance, and 4 indicates the healthcare provider is strongly satisfied with implant performance. In still another embodiment, implant performance is measured using a five point scale where 1 indicates the healthcare provider is definitely dissatisfied with implant performance, 2 indicates the healthcare provider is somewhat dissatisfied with implant performance, 3 indicates the healthcare provider is neither dissatisfied nor satisfied with implant performance, 4 indicates the healthcare provider is somewhat satisfied with implant performance, and 5 indicates the healthcare provider is definitely satisfied with implant performance.

In some embodiments, a physical examination assessment includes evaluating satisfaction of an individual with the implant on its performance may be measured using a questionnaire. The questionnaire can contain questions relating to, for example but without limitation, patient satisfaction with the implant itself, satisfaction with the medical care received, over-all well-being after implantation, psychological or psychosocial well-being, and satisfaction with over-all outcome of the implant. In some embodiments, the questionnaire can also contain additional questions specifically tailored to the type of implant, such as, for example in the case of breast augmentation, sexual well-being. One example of such a questionnaire that may be used in the case of breast implants is the BREAST-Q. See, e.g., A. L. Pusic, et al., Plast. Reconstr. Surg. 124: 345-353 (2009), which is incorporated herein by reference in its entirety.

Evaluating satisfaction of individual with the implant on its performance may be measured using a scale to quantify the subjective assessment of the individual. In one embodiment, implant performance is measured using a two point scale where 1 indicates the individual is dissatisfied with implant performance and 2 indicates the individual is satisfied with implant performance. In another embodiment, implant performance is measured using a three point scale where 1 indicates the individual is dissatisfied with implant performance, 2 indicates the individual is neither dissatisfied nor satisfied with implant performance, and 3 indicates the individual is definitely satisfied with implant performance. In yet another embodiment, implant performance is measured using a four point scale where 1 indicates the individual is strongly dissatisfied with implant performance, 2 indicates the individual is somewhat dissatisfied with implant performance, 3 indicates the individual is somewhat satisfied with implant performance, and 4 indicates the individual is strongly satisfied with implant performance. In still another embodiment, implant performance is measured using a five point scale where 1 indicates the individual is definitely dissatisfied with implant performance, 2 indicates the individual is somewhat dissatisfied with implant performance, 3 indicates the individual is neither dissatisfied nor satisfied with implant performance, 4 indicates the individual is somewhat satisfied with implant performance, and 5 indicates the individual is definitely satisfied with implant performance.

In some embodiments, a physical examination assessment includes evaluating whether the implant is palpably distinguishable from surrounding tissue. Such evaluation may be done by a healthcare provider or the individual with the implant. Evaluating whether an implant is palpably distinguishable from surrounding tissue may be measured using a scale to quantify the subjective assessment of the observer. In one embodiment, evaluating whether an implant is palpably distinguishable from surrounding tissue is measured using a two-point scale where 1 indicates the implanted device is distinguishable from the surrounding tissue, and 2 indicates the implanted device is indistinguishable from the surrounding tissue. In another embodiment, evaluating whether an implant is palpably distinguishable from surrounding tissue is measured using a three-point scale where 1 indicates the implanted device is easy to distinguish from the surrounding tissue, 2 indicates the implanted device is difficult to distinguish from the surrounding tissue, and 3 indicates that the implanted device is indistinguishable from the surrounding tissue. In yet another embodiment, evaluating whether an implant is palpably distinguishable from surrounding tissue is measured using a four-point scale where 1 indicates the implanted device is very easy to distinguish from the surrounding tissue, 2 indicates the implanted device is somewhat easy to distinguish from the surrounding tissue, 3 indicates the implanted device is somewhat difficult to distinguish from the surrounding tissue, and 4 indicates the implanted device is very difficult to distinguish from the surrounding tissue. In still another embodiment, evaluating whether an implant is palpably distinguishable from surrounding tissue is measured using a five-point scale where 1 indicates the implanted device is very easy to distinguish from the surrounding tissue, 2 indicates the implanted device is somewhat easy to distinguish from the surrounding tissue, 3 indicates the implanted device is somewhat difficult to distinguish from the surrounding tissue, 4 indicates the implanted device is very difficult to distinguish from the surrounding tissue, and 5 indicates the implanted device is indistinguishable from the surrounding tissue.

In some embodiments, a physical examination assessment includes evaluating whether capsular contraction is present or absent. Such evaluation may be done by a healthcare provider or the individual with the implant. Evaluating whether capsular contraction is present or absent may be measured using a scale to quantify the subjective assessment of the observer. In one embodiment, evaluating the presence or absence of capsular contraction is measured using a two-point scale wherein 1 indicates the presence of capsular contraction, and 2 indicates the absence of capsular contraction. In another embodiment, evaluating the presence or absence of capsular contraction is measured using a four-point scale wherein 1 indicates the surrounding tissue is soft and normal in appearance, 2 indicates the surrounding tissue is firm but normal in appearance, 3 indicates the surrounding tissue is firm and abnormal in appearance, and 4 indicates the surrounding tissue is firm and abnormal in appearance and the individual is experiencing pain.

In some embodiments of the present disclosure, the scale for assessing the presence or absence of capsular contracture comprises the four-grade Baker scale (also known as the Baker Grade assessment, Baker Grade classification, or Baker Grade System), an assessment based on the look and feel of the tissue surrounding the implant. In the example of breast implants, a Baker Grade I means the breast is normally soft and appears natural in size and shape; Grade II means the breast is somewhat firm, but appears normal; Grade III means the breast is firm and appears abnormal; and, Grade IV means the breast is hard, painful to the individual when touched, and appears abnormal and distorted in shape.

A qualitative assessment by photography may include, without limitation, still photography, video recordings, thermal imaging. An evaluator analysis the obtained pictures by assessing for an occurrence and/or extent capsular formation and/or an occurrence and/or extent capsular contracture. Such evaluation may be done by a healthcare provider or the individual with the implant.

In some embodiments, this photographic assessment comprises one or more pictures of the surface area of the individual with the implant in the vicinity of the implanted device. In some embodiments, the pictures can include one or more of a frontal view of the individual, a left-side lateral view of the individual, a right-side lateral view of the individual, or any combination thereof. In aspects of these embodiments, the implant is a breast implant, and the photographic assessment can include one or more photographs comprising a frontal view of the individual with hands at rest by sides, a frontal view of the individual with hands at rest on waist, a frontal view of the individual with hands firmly pressing down on waist, a left-side lateral view of the individual with hands at rest by sides, a right-side lateral view of the individual with hands at rest by sides, a left-side 45-degree diagonal view of the individual with hands at rest by sides, a right-side 45-degree diagonal view of the individual with hands at rest by sides, a view of the individual leaning forward towards the camera with hands at rest on waist, or any combination thereof.

Examples of quantitative assessments useful for assessing an occurrence and/or extent capsular formation and/or an occurrence and/or extent capsular contracture may include, without limitation, depth measurements, tissue integration measurements, tonometry measurements, anatomical measurements, or any combination thereof.

A quantitative assessment by a depth measurement determines a distance between two landmarks locations, one of which is internal to the body. Generally, the greater a change in a distance of a depth measurement relative to a depth baseline measurement, the greater the indication that capsular formation occurred and to what extent and/or the greater the indication that capsular contraction occurred and to what extent. A depth baseline measurement may be an averaged distance based upon a depth measurement taken from a population of individuals, an averaged distance based upon a plurality of previous depth measurements taken from the same individual, a distance based upon one previous depth measurement taken from the same individual, or any other depth measurement distance obtained that is statistically relevant and/or provides a meaningful comparison. Any method that can determine the distance between two landmarks locations can be useful in determining a depth measurement. Non-limiting examples include ultrasound, high resolution ultrasound (HRUS), thermal imaging, radiograph, and sonagraph.

In aspects of this embodiment, a change in a depth measurement of, e.g., 0.2 mm or more, 0.4 mm or more, 0.6 mm or more, 0.8 mm or more, 1 mm or more, 1.2 mm or more, 1.4 mm or more, 1.6 mm or more, 1.8 mm or more, 2.0 mm or more, 2.5 mm or more, 3.0 mm or more, 3.5 mm or more, 4.0 mm or more, 4.5 mm or more, 5.0 mm or more, 5.5 mm or more, 6.0 mm or more, 6.5 mm or more, 7.0 mm or more, 7.5 mm or more, 8.0 mm or more, 8.5 mm or more, 9.0 mm or more, 9.5 mm or more, or 10 mm or more, relative to a depth baseline measurement is indicative of an occurrence and/or extent of capsular formation, indicative of an occurrence and/or extent of capsular contraction, or any combination thereof.

In other aspects of this embodiment, a change in a depth measurement of between, e.g., about 0.2 mm to about 1.0 mm, about 0.2 mm to about 1.4 mm, about 0.2 mm to about 1.8 mm, about 0.2 mm to about 2.2 mm, about 0.2 mm to about 2.4 mm, about 0.2 mm to about 2.8 mm, about 0.2 mm to about 3.2 mm, about 0.4 mm to about 1.0 mm, about 0.4 mm to about 1.4 mm, about 0.4 mm to about 1.8 mm, about 0.4 mm to about 2.2 mm, about 0.4 mm to about 2.4 mm, about 0.4 mm to about 2.8 mm, about 0.4 mm to about 3.2 mm, about 0.6 mm to about 1.0 mm, about 0.6 mm to about 1.4 mm, about 0.6 mm to about 1.8 mm, about 0.6 mm to about 2.2 mm, about 0.6 mm to about 2.4 mm, about 0.6 mm to about 2.8 mm, about 0.6 mm to about 3.2 mm, about 0.8 mm to about 1.0 mm, about 0.8 mm to about 1.4 mm, about 0.8 mm to about 1.8 mm, about 0.8 mm to about 2.2 mm, about 0.8 mm to about 2.4 mm, about 0.8 mm to about 2.8 mm, about 0.8 mm to about 3.2 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1 mm to about 5.5 mm, about 1 mm to about 6 mm, about 1 mm to about 6.5 mm, about 1 mm to about 7 mm, about 1 mm to about 7.5 mm, about 1 mm to about 8 mm, about 1 mm to about 8.5 mm, about 1 mm to about 9 mm, about 1 mm to about 9.5 mm, or about 1 mm to about 10 mm, relative to a depth baseline measurement is indicative of an occurrence and/or extent of capsular formation, indicative of an occurrence and/or extent of capsular contraction, or any combination thereof.

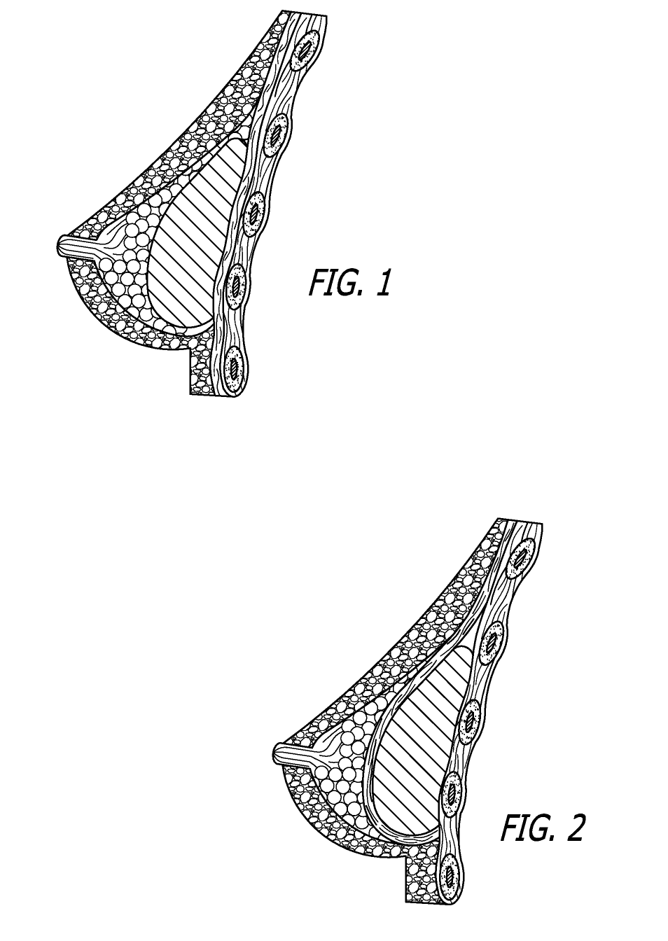

Various types of depth measurements may be obtained according to the present disclosure including, without limitation, the distance from the skin surface and a landmark feature of the implanted device, the distance from the skin surface and a landmark feature of an internal anatomical structure, the distance between two internal anatomical structures, the distance from an internal anatomical structure and a landmark feature of the implanted device, or any combination thereof. In some embodiments, a depth measurement is a distance from the skin surface to the outside surface of the implant (FIG. 1). In some embodiments, a depth measurement is a distance from the skin surface to a tissue layer directly overlying the implanted device (FIG. 1). In some embodiments, a depth measurement is a distance from the skin surface to a tissue layer directly underlying the implanted device (FIG. 2). A tissue layer includes, without limitation, a glandular layer, a muscle layer, a hypodermal layer, and a bone layer. In some embodiments, a depth measurement is a thickness of a formed capsule.

In aspects of these embodiments, a thickness of a formed capsule of, e.g., 25 μm or more, 50 μm or more, 75 μm or more, 100 μm or more, 125 μm or more, 150 μm or more, 175 μm or more, or 200 μm or more, is indicative of an occurrence and/or extent of capsular formation, indicative of an occurrence and/or extent of capsular contraction, or any combination thereof. In other aspects of these embodiments, a thickness of a formed capsule of between, e.g., about 25 μm to about 100 μm, about 25 μm to about 150 μm, about 25 μm to about 200 μm, about 25 μm to about 250 μm, about 25 μm to about 300 μm, about 50 μm to about 100 μm, about 50 μm to about 150 μm, about 50 μm to about 200 μm, about 50 μm to about 250 μm, about 50 μm to about 300 μm, about 75 μm to about 100 μm, about 75 μm to about 150 μm, about 75 μm to about 200 μm, about 75 μm to about 250 μm, about 75 μm to about 300 μm, about 100 μm to about 150 μm, about 100 μm to about 200 μm, about 100 μm to about 250 μm, about 100 μm to about 300 μm, about 100 μm to about 350 μm, about 100 μm to about 400 μm, about 125 μm to about 150 μm, about 125 μm to about 200 μm, about 125 μm to about 250 μm, about 125 μm to about 300 μm, about 125 μm to about 350 μm, about 125 μm to about 400 μm, about 150 μm to about 200 μm, about 150 μm to about 250 μm, about 150 μm to about 300 μm, about 150 μm to about 350 μm, or about 150 μm to about 400 μm, is indicative of an occurrence and/or extent of capsular formation, indicative of an occurrence and/or extent of capsular contraction, or any combination thereof.

A quantitative assessment by a tissue integration measurement determines the degree of cellular ingrowth in and around implanted device. Generally, the higher the degree of cellular ingrowth in and around implanted device, the greater the indication that capsular formation occurred and to what extent and/or the greater the indication that capsular contraction occurred and to what extent. Any method that can determine the extent or degree of cellular ingrowth in and around implanted device can be useful in determining a tissue integration measurement. Non-limiting examples include ultrasound, high resolution ultrasound (HRUS), thermal imaging, radiograph, and sonagraph. Typically, a tissue integration measurement is performed when the implanted device has a textured surface of some kind.

In some embodiments, a tissue integration measurement determines the planar and directional orientation of cells and/or tissue in and around implanted device as an indicia of assessing an occurrence and/or extent capsular formation and/or an occurrence and/or extent capsular contracture. The cells may be fibroblasts.

In aspects of these embodiments, a cell infiltration into the implant surface of, e.g., 25 μm or less, 50 μm or less, 75 μm or less, or 100 μm or less, is indicative of an occurrence and/or extent of capsular formation, indicative of an occurrence and/or extent of capsular contraction, or any combination thereof. In other aspects of these embodiments, a cell infiltration into the implant surface of between, e.g., about 1 μm to about 20 μm, about 1 μm to about 30 μm, about 1 μm to about 40 μm, about 1 μm to about 50 μm, about 1 μm to about 60 μm, about 1 μm to about 70 μm, about 1 μm to about 80 μm, about 1 μm to about 90 μm, about 1 μm to about 100 μm, about 5 μm to about 20 μm, about 5 μm to about 30 μm, about 5 μm to about 40 μm, about 5 μm to about 50 μm, about 5 μm to about 60 μm, about 5 μm to about 70 μm, about 5 μm to about 80 μm, about 5 μm to about 90 μm, about 5 μm to about 100 μm, about 10 μm to about 20 μm, about 10 μm to about 30 μm, about 10 μm to about 40 μm, about 10 μm to about 50 μm, about 10 μm to about 60 μm, about 10 μm to about 70 μm, about 10 μm to about 80 μm, about 10 μm to about 90 μm, or about 10 μm to about 100 μm, is indicative of an occurrence and/or extent of capsular formation, indicative of an occurrence and/or extent of capsular contraction, or any combination thereof.

In other aspects of these embodiments, a fibroblast infiltration into the implant surface of, e.g., 25 μm or less, 50 μm or less, 75 μm or less, or 100 μm or less, is indicative of an occurrence and/or extent of capsular formation, indicative of an occurrence and/or extent of capsular contraction, or any combination thereof. In yet other aspects of these embodiments, a fibroblast infiltration into the implant surface of between, e.g., about 1 μm to about 20 μm, about 1 μm to about 30 μm, about 1 μm to about 40 μm, about 1 μm to about 50 μm, about 1 μm to about 60 μm, about 1 μm to about 70 μm, about 1 μm to about 80 μm, about 1 μm to about 90 μm, about 1 μm to about 100 μm, about 5 μm to about 20 μm, about 5 μm to about 30 μm, about 5 μm to about 40 μm, about 5 μm to about 50 μm, about 5 μm to about 60 μm, about 5 μm to about 70 μm, about 5 μm to about 80 μm, about 5 μm to about 90 μm, about 5 μm to about 100 μm, about 10 μm to about 20 μm, about 10 μm to about 30 μm, about 10 μm to about 40 μm, about 10 μm to about 50 μm, about 10 μm to about 60 μm, about 10 μm to about 70 μm, about 10 μm to about 80 μm, about 10 μm to about 90 μm, or about 10 μm to about 100 μm, is indicative of an occurrence and/or extent of capsular formation, indicative of an occurrence and/or extent of capsular contraction, or any combination thereof.

In aspects of these embodiments, a degree of cell infiltration of, e.g., 10% or less, 20% or less, 30% or less 40% or less, 50% or less, 60% or less, of cells infiltrating into the implanted device is indicative of an occurrence and/or extent of capsular formation, indicative of an occurrence and/or extent of capsular contraction, or any combination thereof. In other aspects of these embodiments, a degree of cell infiltration of, e.g., about 1% to about 10%, about 1% to about 20%, about 1% to about 30%, about 1% to about 40%, about 1% to about 50%, about 1% to about 60%, about 5% to about 10%, about 5% to about 20%, about 5% to about 30%, about 5% to about 40%, about 5% to about 50%, about 5% to about 60%, about 10% to about 20%, about 10% to about 30%, about 10% to about 40%, about 10% to about 50%, about 10% to about 60%, about 15% to about 20%, about 15% to about 30%, about 15% to about 40%, about 15% to about 50%, or about 15% to about 60%, of cells infiltrating into the implanted device is indicative of an occurrence and/or extent of capsular formation, indicative of an occurrence and/or extent of capsular contraction, or any combination thereof.

In aspects of these embodiments, a degree of fibroblast infiltration of, e.g., 10% or less, 20% or less, 30% or less 40% or less, 50% or less, 60% or less, of fibroblasts infiltrating into the implanted device is indicative of an occurrence and/or extent of capsular formation, indicative of an occurrence and/or extent of capsular contraction, or any combination thereof. In other aspects of these embodiments, a degree of fibroblast infiltration of, e.g., about 1% to about 10%, about 1% to about 20%, about 1% to about 30%, about 1% to about 40%, about 1% to about 50%, about 1% to about 60%, about 5% to about 10%, about 5% to about 20%, about 5% to about 30%, about 5% to about 40%, about 5% to about 50%, about 5% to about 60%, about 10% to about 20%, about 10% to about 30%, about 10% to about 40%, about 10% to about 50%, about 10% to about 60%, about 15% to about 20%, about 15% to about 30%, about 15% to about 40%, about 15% to about 50%, or about 15% to about 60%, of fibroblasts infiltrating into the implanted device is indicative of an occurrence and/or extent of capsular formation, indicative of an occurrence and/or extent of capsular contraction, or any combination thereof.

In other aspects of these embodiments, a degree of cell orientation of, e.g., 10% or more, 20% or more, 30% or more 40% or more, 50% or more, 60% or more, 70% or more, 80% or more, or 90% or more of cells not orientated in parallel to the implant surface is indicative of an occurrence and/or extent of capsular formation, indicative of an occurrence and/or extent of capsular contraction, or any combination thereof. In yet other aspects of these embodiments, a degree of cell orientation of, e.g., about 10% to about 20%, about 10% to about 30%, about 10% to about 40%, about 20% to about 30%, about 20% to about 40%, about 20% to about 50%, about 30% to about 40%, about 30% to about 50%, about 30% to about 60%, about 40% to about 50%, about 40% to about 60%, about 40% to about 70%, about 50% to about 60%, about 50% to about 70%, about 50% to about 80%, about 60% to about 70%, about 60% to about 80%, about 60% to about 90%, about 70% to about 80%, about 70% to about 90%, about 70% to about 100% of cells not orientated in parallel to the implant surface is indicative of an occurrence and/or extent of capsular formation, indicative of an occurrence and/or extent of capsular contraction, or any combination thereof.

In other aspects of these embodiments, a degree of fibroblast orientation of, e.g., 10% or more, 20% or more, 30% or more 40% or more, 50% or more, 60% or more, 70% or more, 80% or more, or 90% or more of fibroblasts not orientated in parallel to the implant surface is indicative of an occurrence and/or extent of capsular formation, indicative of an occurrence and/or extent of capsular contraction, or any combination thereof. In yet other aspects of these embodiments, a degree of fibroblast orientation of, e.g., about 10% to about 20%, about 10% to about 30%, about 10% to about 40%, about 20% to about 30%, about 20% to about 40%, about 20% to about 50%, about 30% to about 40%, about 30% to about 50%, about 30% to about 60%, about 40% to about 50%, about 40% to about 60%, about 40% to about 70%, about 50% to about 60%, about 50% to about 70%, about 50% to about 80%, about 60% to about 70%, about 60% to about 80%, about 60% to about 90%, about 70% to about 80%, about 70% to about 90%, about 70% to about 100% of fibroblasts not orientated in parallel to the implant surface is indicative of an occurrence and/or extent of capsular formation, indicative of an occurrence and/or extent of capsular contraction, or any combination thereof.

A quantitative assessment by a tonometry measurement determines a tissue pressure calculated from an applied force. Generally, the higher the measured tissue pressure, the greater the indication that capsular formation occurred and to what extent and/or the greater the indication that capsular contraction occurred and to what extent.

In some embodiment, a tissue pressure having a peak force of, e.g., 1 N or more, 2 N or more, 3 N or more, 4 N or more, 5 N or more, 6 N or more, 7 N or more, 8 N or more, 9 N or more, 10 N or more, 11 N or more, is indicative of an occurrence and/or extent of capsular formation, indicative of an occurrence and/or extent of capsular contraction, or any combination thereof. In some embodiments, a tissue pressure having a peak force of between, e.g., about 6 N to about 8 N, about 6 N to about 9 N, about 6 N to about 10 N, about 6 N to about 11 N, about 7 N to about 9 N, about 7 N to about 10 N, about 7 N to about 11 N, about 7 N to about 12 N, about 8 N to about 10 N, about 8 N to about 11 N, about 8 N to about 12 N, about 8 N to about 13 N, about 9 N to about 11 N, about 9 N to about 12 N, about 9 N to about 13 N, about 9 N to about 14 N, about 10 N to about 12 N, about 10 N to about 13 N, about 10 N to about 14 N, about 10 N to about 15 N, about 11 N to about 13 N, about 11 N to about 14 N, about 11 N to about 15 N, or about 11 N to about 16 N, is indicative of an occurrence and/or extent of capsular formation, indicative of an occurrence and/or extent of capsular contraction, or any combination thereof.

In some embodiment, a tissue pressure having a peak force of, e.g., 1 N or less, 2 N or less, 3 N or less, 4 N or less, 5 N or less, 6 N or less, 7 N or less, 8 N or less, 9 N or less, 10 N or less, 11 N or less, is indicative of an occurrence and/or extent of capsular formation, indicative of an occurrence and/or extent of capsular contraction, or any combination thereof. In some embodiments, a tissue pressure having a peak force of between, e.g., about 1 N to about 5 N, about 1 N to about 6 N, about 1 N to about 7 N, about 1 N to about 8 N, about 1 N to about 9 N, about 1 N to about 10 N, about 2 N to about 5 N, about 2 N to about 6 N, about 2 N to about 7 N, about 2 N to about 8 N, about 2 N to about 9 N, about 2 N to about 10 N, about 3 N to about 5 N, about 3 N to about 6 N, about 3 N to about 7 N, about 3 N to about 8 N, about 3 N to about 9 N, about 3 N to about 10 N, about 4 N to about 5 N, about 4 N to about 6 N, about 4 N to about 7 N, about 4 N to about 8 N, about 4 N to about 9 N, about 4 N to about 10 N, is indicative of an occurrence and/or extent of capsular formation, indicative of an occurrence and/or extent of capsular contraction, or any combination thereof.

In some embodiment, a tissue pressure having a stiffness of, e.g., 20 mmHg/mL or more, 25 mmHg/mL or more, 30 mmHg/mL or more, 35 mmHg/mL or more, 40 mmHg/mL or more, 45 mmHg/mL or more, or 50 mmHg/mL or more, is indicative of an occurrence and/or extent of capsular formation, indicative of an occurrence and/or extent of capsular contraction, or any combination thereof. In some embodiment, a tissue pressure having a stiffness of, e.g., about 20 mmHg/mL to about 30 mmHg/mL, about 20 mmHg/mL to about 40 mmHg/mL, about 20 mmHg/mL to about 50 mmHg/mL, about 20 mmHg/mL to about 60 mmHg/mL, about 25 mmHg/mL to about 30 mmHg/mL, about 25 mmHg/mL to about 40 mmHg/mL, about 25 mmHg/mL to about 50 mmHg/mL, about 25 mmHg/mL to about 60 mmHg/mL, about 30 mmHg/mL to about 40 mmHg/mL, about 30 mmHg/mL to about 50 mmHg/mL, or about 30 mmHg/mL to about 60 mmHg/mL, is indicative of an occurrence and/or extent of capsular formation, indicative of an occurrence and/or extent of capsular contraction, or any combination thereof.

Various types of tonometry measurements may be obtained according to the present disclosure including, without limitation, applanation tonometry (AT) and/or indentation (or impression) tonometry (IT).

In some embodiments, the extent of capsular contraction can be measured using AT. AT is a quantitative measurement of tissue pressure calculated from the force required to applanate (i.e., flatten) a constant area of the skin surface over the tissue of interest. In some embodiments, the AT measurement can be calculated using the formula P=F/A, where P is the pressure (tonometry) measurement, F is the amount of force applied to the skin surface (e.g., in Newtons), and A is the surface area to which the force is applied and which is flattened in the tonometry measurement. In AT, the higher the tissue pressure, the more force required to applanate a surface area. Conversely, the lower the tissue pressure, the less force required to applanate a surface area.

In some embodiments, the extent of capsular contraction can be measured using IT. IT is a quantitative measurement of tissue pressure calculated from the depth of indentation in the skin surface above the tissue of interest made by a small probe or plunger possessing a known weight. Typically, the probe is attached to a device used to measure force applied to the skin surface (e.g., in Newtons). In IT, the higher the tissue pressure, the more shallow the indentation made by the applied force exerted using the probe. Conversely, the lower the tissue pressure, the more deep the indentation made by the applied force exerted using the probe.

An indentation measurement may be made in one location directly overlying the implanted device, in a plurality of locations directly overlying the implanted device, one location directly overlying the implanted device and in one location not directly overlying the implanted device, in a plurality of locations directly overlying the implanted device and in one location not directly overlying the implanted device, and in a plurality of locations directly overlying the implanted device and a plurality of locations not directly overlying the implanted device. Generally speaking, indentation measurement made in locations directly overlying the implanted device are used to measure the extent of capsular contraction, whereas indentation measurement made in locations not directly overlying the implanted device are used as control measurements.

In aspects of this embodiment, indentation measurements are made in, e.g., at least two locations directly overlying the implanted device, at least three locations directly overlying the implanted device, at least four locations directly overlying the implanted device, at least five locations directly overlying the implanted device, at least six locations directly overlying the implanted device, or at least seven locations directly overlying the implanted device. In other aspects of this embodiment, indentation measurements are made in, e.g., about two to about four locations directly overlying the implanted device, about two to about five locations directly overlying the implanted device, about two to about six locations directly overlying the implanted device, about two to about seven locations directly overlying the implanted device, about two to about eight locations directly overlying the implanted device, about two to about nine locations directly overlying the implanted device, about two to about ten locations directly overlying the implanted device, about three to about four locations directly overlying the implanted device, about three to about five locations directly overlying the implanted device, about three to about six locations directly overlying the implanted device, about three to about seven locations directly overlying the implanted device, about three to about eight locations directly overlying the implanted device, about three to about nine locations directly overlying the implanted device, about three to about ten locations directly overlying the implanted device, about four to about five locations directly overlying the implanted device, about four to about six locations directly overlying the implanted device, about four to about seven locations directly overlying the implanted device, about four to about eight locations directly overlying the implanted device, about four to about nine locations directly overlying the implanted device, or about four to about ten locations directly overlying the implanted device.

When taking indentation measurement from a plurality of locations, the locations may be determined randomly or based on a geometric pattern of some sort. In aspects of this embodiment, indentation measurements may be taken from the 2 o'clock, 6 o'clock, and 10 o'clock positions based on the central location of the implanted device. In other aspects of this embodiment, indentation measurements may be taken from the 3 o'clock, 6 o'clock, 9 o'clock, and 12 o'clock positions based on the central location of the implanted device. In yet other aspects of this embodiment, indentation measurements may be taken from the 2 o'clock, 4 o'clock, 6 o'clock, 8 o'clock, 10 o'clock, and 12 o'clock positions based on the central location of the implanted device.

In some embodiments, tonometry can be simply measured by palpation; i.e., by the healthcare provider pressing on the skin surface to determine intratissue pressure. This example of tonometry is a qualitative assessment because the assessment as to the extent of tonometry does not come from a numerically measured value, but instead depends on the subjective judgment of the healthcare provider.

A quantitative assessment by an anatomical measurement determines a distance between two anatomical feature observable from the skin surface. Generally, the greater a change in a distance of an anatomical measurement relative to an anatomical baseline measurement, the greater the indication that capsular formation occurred and to what extent and/or the greater the indication that capsular contraction occurred and to what extent. An anatomical baseline measurement may be an averaged distance based upon an anatomical measurement taken from a population of individuals, an averaged distance based upon a plurality of previous anatomical measurements taken from the same individual, a distance based upon one previous anatomical measurement taken from the same individual, or any other anatomical measurement distance obtained that is statistically relevant and/or provides a meaningful comparison.

Typically, an anatomical measurement comprises one or more surface measurements of anatomical features in the vicinity of the implanted device. In embodiments wherein the implanted device is a breast implant, an anatomical measurement may comprise a breast base width distance, breast height distance, breast fold line to nipple projection position distance, breast to breast distance, nipple to inframammary fold distance, nipple to sternal notch distance, nipple to nipple distance, nipple to midline distance, nipple to mid-clavicle distance, areolar diameter, or any combination thereof.

In aspects of this embodiment, a change in an anatomical measurement of, e.g., 0.5 mm or more, 1.0 mm or more, 1.5 mm or more, 2.0 mm or more, 2.5 mm or more, 3.0 mm or more, 3.5 mm or more, 4.0 mm or more, 4.5 mm or more, 5.0 mm or more, 5.5 mm or more, 6.0 mm or more, 6.5 mm or more, 7.0 mm or more, 7.5 mm or more, 8.0 mm or more, 8.5 mm or more, 9.0 mm or more, 9.5 mm or more, 10 mm or more, 15 mm or more, 20 mm or more, 25 mm or more, 30 mm or more, 35 mm or more, 40 mm or more, 45 mm or more, 50 mm or more, relative to an anatomical baseline measurement is indicative of an occurrence and/or extent of capsular formation, indicative of an occurrence and/or extent of capsular contraction, or any combination thereof.

In aspects of this embodiment, a change in an anatomical measurement of between, e.g., about 1 mm to about 5 mm, about 1 mm to about 10 mm, about 1 mm to about 15 mm, about 1 mm to about 20 mm, 5 mm to about 10 mm, about 5 mm to about 15 mm, about 5 mm to about 20 mm, about 5 mm to about 25 mm, about 10 mm to about 15 mm, about 10 mm to about 20 mm, about 10 mm to about 25 mm, about 10 mm to about 30 mm, about 15 mm to about 20 mm, about 15 mm to about 25 mm, about 15 mm to about 30 mm, about 15 mm to about 35 mm, about 20 mm to about 25 mm, about 20 mm to about 30 mm, about 20 mm to about 35 mm, about 20 mm to about 40 mm, about 25 mm to about 30 mm, about 25 mm to about 35 mm, about 25 mm to about 40 mm, about 25 mm to about 45 mm, about 30 mm to about 35 mm, about 30 mm to about 40 mm, about 30 mm to about 45 mm, about 30 mm to about 50 mm, relative to a baseline measurement is indicative of an occurrence and/or extent of capsular formation, indicative of an occurrence and/or extent of capsular contraction, or any combination thereof.

In some embodiments, methods for assessing the extent of capsular contraction around an implanted device in an individual do not include a physical examination assessment. In other embodiments, the methods comprise obtaining a physical examination assessment and one or more of assessments of the area in the vicinity of the implanted device, the one or more assessments comprising a first depth measurement from a skin surface of the individual, a second depth measurement from the skin surface of the individual, a tissue integration measurement of surrounding tissue with the implanted device, a tonometry measurement, an anatomical measurement, a photographic measurement, or any combination thereof.

In other embodiments, methods comprise obtaining one or more quantitative assessments of the area in the vicinity of the implanted device. In some embodiments the quantitative assessments comprise a first depth measurement from a skin surface of the individual, a second depth measurement from the skin surface of the individual, an integration measurement of surrounding tissue with the implanted device, a tonometry measurement, an anatomical measurement, or any combination thereof. In some embodiments of the present disclosure, the methods do not include a qualitative assessment.

In some embodiments, when the implant is a breast implant, methods disclosed herein comprise obtaining one or more assessments of the area in the vicinity of the breast implant comprising an ultrasound measurement, a tonometry measurement, a physical examination assessment, an anatomical measurement, a photographic assessment, or any combination thereof. In some embodiments, when the implant is a breast implant, methods disclosed herein comprise a physical examination assessment which includes evaluating satisfaction of a healthcare provider with the breast implant, evaluating satisfaction of the individual with the breast implant, determining whether the breast implant is palpably distinguishable from surrounding tissue, and/or evaluating presence of capsular contraction. In some embodiments wherein the implant is a breast implant, the methods comprise obtaining a physical examination assessment and one or more assessments of the area in the vicinity of the breast implant, the one or more assessments comprising an ultrasound measurement, a tonometry measurement, another physical examination assessment, an anatomical measurement, a photographic assessment, or any combination thereof.

Aspects of the present specification relate to methods for monitoring capsular formation and/or capsular contracture around an implanted device over time. Such methods may include obtaining one or more assessments as described herein at a plurality of time points before, at, and/or after placement of the implant. In aspects of this embodiment, methods for monitoring capsular formation and/or capsular contracture around an implanted device over time may include obtaining one or more assessments as described herein before implantation and obtaining one or more assessments as described herein after implantation. In other aspects of this embodiment, methods for monitoring capsular formation and/or capsular contracture around an implanted device over time may include obtaining one or more assessments as described herein at implantation and obtaining one or more assessments as described herein after implantation. In aspects of this embodiment, methods for monitoring capsular formation and/or capsular contracture around an implanted device over time may include obtaining one or more assessments as described herein before implantation, obtaining one or more assessments as described herein at implantation, and obtaining one or more assessments as described herein after implantation. By comparing the assessments at multiple time points, discernment on whether capsular formation is occurring and to what extent and/or whether capsular contraction is occurring and to what extent can be achieved. Such monitoring of the formation and/or progressive development of capsular formation and/or capsular contracture over time provides many benefits, including, without limitation, determining whether palliative measures, reoperation or explanation of the device is advisable.

In some embodiments, the one or more assessments can by themselves an assessment of contracture. In other embodiments, the one or more assessments can be compared to the same one or more assessments obtained before implantation. In still other embodiments, the one or more assessments can be compared to the same one or more assessments obtained from like individuals with or without an implanted device (the latter being “controls”).

Capsular formation and/or contracture may begin to occur within a wide range of time following implantation of the device. With breast implants, for example, capsular contracture can be observed in the first several months after surgery, but can also be observed years after implantation.

In aspects of this embodiment, methods for monitoring capsular formation and/or capsular contracture formation comprise a first time point wherein one or more assessments as disclosed herein are obtained one week to one month following implantation of the device, and subsequent time points wherein one or more assessments as disclosed herein are obtained at one or more one-month intervals thereafter. In aspects of these embodiments, the one or more assessments as disclosed herein are obtained from the subsequent time points for a period of 3 months, about 4 months, 5 months, 6 months, 7 months, 8 months, 9 months, 10 months, 11 months, 12 months, about 1.5 years, about 2 years, about 2.5 years, or about 3 years. In other aspects of these embodiments, one or more assessments are obtained from the first time point at two months, three months, four months, five months, or six months after implantation.

In some embodiments, methods for monitoring capsular formation and/or capsular contracture formation comprise a first time point wherein one or more assessments as disclosed herein are obtained one week to one month following implantation of the device, and subsequent time points wherein one or more assessments as disclosed herein are obtained at one or more three-month intervals thereafter. In aspects of these embodiments, the one or more assessments as disclosed herein are obtained from the subsequent time points for a period of about 6 months, about 9 months, about 1 year, about 1.5 years, about 2 years, about 2.5 years, about 3 years, about 3.5 years, about 4 years, about 4.5 years, or about 5 years. In other aspects of these embodiments, one or more assessments are obtained from the first time point at two months, three months, four months, five months, or six months after implantation.

In aspects of this embodiment, methods for monitoring capsular formation and/or capsular contracture formation comprise a first time point wherein one or more assessments as disclosed herein are obtained one week to one month following implantation of the device, and subsequent time points wherein one or more assessments as disclosed herein are obtained at one or more six-month intervals thereafter. In aspects of these embodiments, the one or more assessments as disclosed herein are obtained from the subsequent time points for a period of about 1 year, about 1.5 years, about 2 years, about 2.5 years, about 3 years, about 3.5 years, about 4 years, about 4.5 years, about 5 years, about 5.5 year, about 6 years, about 6.5 years, about 7 years, about 7.5 years, about 8 years, about 8.5 years, about 9 years, about 9.5 years, or about 10 years. In other aspects of these embodiments, one or more assessments are obtained from the first time point at two months, three months, four months, five months, or six months after implantation.

In aspects of this embodiment, methods for monitoring capsular formation and/or capsular contracture formation comprise a first time point wherein one or more assessments as disclosed herein are obtained one week to one month following implantation of the device, and subsequent time points wherein one or more assessments as disclosed herein are obtained at one or more yearly intervals thereafter. In aspects of these embodiments, the one or more assessments as disclosed herein are obtained from the subsequent time points for a period of about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years. In other aspects of these embodiments, one or more assessments are obtained from the first time point at two months, three months, four months, five months, or six months after implantation.

### EXAMPLE

The following example illustrates a representative embodiment now contemplated, but should not be construed to limit the disclosed methods of assessing the extent of capsular contracture or the methods of monitoring capsular contracture formation.

### Example 1

### A Method of Assessing Capsular Contracture

This example illustrates methods of assessing fibrous capsular contracture surrounding an implanted device according to the present disclosure.

In a prospective study, formation of a fibrous capsule surrounding the implant and the occurrence of capsular contracture as well as investigator and subject satisfaction are assessed in women receiving textured, round breast implants for breast augmentation. The purpose of the study is to gain investigator and subject feedback on the device and to characterize the fibrous capsule surrounding the study breast implants and determine the rate of capsular contracture. One objective is to evaluate the Investigator's overall satisfaction with the device at 3 months, 6 months and 2 years post-implantation surgery. Other objectives are to evaluate subject satisfaction with her breasts at 6 months post-implantation surgery, investigator evaluation of whether the implant is palpably distinguishable from the tissue at 3 months, 6 months and 2 years post-implantation, and the presence of capsular contracture at 1 year, 2 years, and 5 years post-implantation. Other objectives are to evaluate: overall Investigator and subject satisfaction; the Investigator's assessment of the ease of use of the implant, its clinical performance, and other characteristics of the surgery in relation to previous similar surgeries performed; capsule formation and thickness; device-tissue integration; capsule firmness; and degree of capsular contracture.

The study is a prospective, multi-center, single-arm clinical study. Fifty subjects are implanted with the device, all subjects being normal, healthy adult females seeking bilateral breast augmentation. The study spans a total of approximately 64 months: an estimated 4 months for recruitment and 60 months (5 years) for follow-up after implantation. Each subject's participation encompasses 5 years of follow-up after device implantation.

The breast implants used in the study are manufactured to result in a unique surface topology according to the present disclosure, designed to increase ingrowth of the implant into host tissue and thus promote successful integration with host tissue. The implants are silicone-filled breast implants with a silicone surface texture containing interconnected pores (similar in structure to polyurethane textured surfaces), intended to provide a deeper substrate for tissue integration following implantation. The manufacturing process as described herein results in a thicker texturing on the outer aspect of the shell with multiple interconnected porous layers to allow for increased tissue integration.

Detection methods that are used to confirm implant capsular formation and assess severity of contracture include high resolution ultrasound (HRUS) imaging, breast firmness measurements of applanation tonometry (AT) and indentation tonometry (IT), and the subjective assessment of Baker Grade classification, which is based on breast morphology and discomfort assessment. The techniques used in this study to evaluate capsule formation, assess investigator satisfaction with the device during and after implantation, and assess subject satisfaction with her breasts and psychosocial well-being, increase understanding regarding whether the unique texture of the study breast implants will lead to an improved risk-benefit profile compared to other textured breast implants.

Before implantation a screening visit is conducted. BREAST-Q questionnaires are completed by the subject prior to conducting other screening visit procedures. Breast examinations are performed according to best clinical practices and include palpation of all quadrants, axillary tail of Spence, and nodes. The subject undergoes standardized photography of her breasts. For baseline assessments, the subject undergoes: (a) HRUS with 2 parameters measured per breast resulting in 16 images collected, including (i) distance from skin surface to innermost (deepest) surface of the gland and (ii) distance from skin surface to innermost (deepest) surface of the pectoralis major muscle, each at 4 sites on each breast; 12 o'clock position, 3 o'clock position, 6 o'clock position, and 9 o'clock position, each at a distance from the nipple equal to half the nipple-inframammary fold distance; (b) AT to determine the overall intramammary pressure (firmness) for each breast; (c) IT to determine intramammary pressure (firmness), in which the average of 3 measurements are determined at 5 specific locations in the breast: a control site located superior to the anticipated superior border of the implant, and at 4 anticipated implant sites (over the gland) at 12 o'clock position, 3 o'clock position, 6 o'clock position, and 9 o'clock position, each at a distance from the nipple equal to half the nipple-inframammary fold distance.

Implant surgery is performed within 4 weeks of the screening visit. Subjects attend follow-up visits at 6 weeks and 3, 6, 9, 12, 18, 24, 36, 48, and 60 months following implant surgery. The Investigator asks the subject a general question as to how she feels since the last visit. BREAST-Q questionnaires are completed by the subject prior to conducting other visit procedures. Breast anatomical measurements and photography of the subject's breasts occur at each follow-up visit. Breast examination according to best clinical practices, including palpation of all quadrants, axillary tail of Spence, and nodes are also performed at all follow-up visits. To monitor for capsular contracture, subjects undergo HRUS with 3 parameters measured: (1) distance from skin surface to inside surface of breast implant shell, (2) distance from skin surface to innermost surface of the gland or muscle tissue that immediately overlies the implant, and (3) dynamic assessment of the presence or absence of tissue integration with the implant at Week 6 and Months 3, 6, 12, 18, and 24; AT to determine the overall intramammary firmness at all follow-up visits; and, IT to determine intramammary firmness at specific locations in the breast at all follow-up visits.

At each follow-up visit, the Investigator also assesses capsular contracture severity using the Baker Grade classification. The Investigator evaluates his/her overall satisfaction with the device as well as satisfaction with regards to use and clinical performance and evaluates overall satisfaction with the device compared to prior experience using 5-point scales. Additionally, the Investigator assesses whether the implant is palpably distinguishable from the tissue and whether the implant provides adequate projection.

Any general adverse events (AEs) as well as breast-specific AEs including asymmetry, breast pain, bruising, capsular contracture, capsule calcification, implant extrusion, implant malposition, implant palpability, implant redness, irritation, other abnormal scarring, palpable orientation marks, skin hypersensitivity, swelling, upper pole fullness or deficiency, visibility, and wrinkling/rippling are recorded.

The primary effectiveness measure is the Investigator's overall satisfaction with the device for each subject using a 5-point scale, where 1 means definitely dissatisfied, 2 means somewhat dissatisfied, 3 means neither satisfied nor dissatisfied, 4 means somewhat satisfied, and 5 means definitely satisfied. Secondary effectiveness measures include: (1) subject satisfaction with breasts as measured by the BREAST-Q questionnaire, (2) presence of capsular contracture defined by Baker Grades III and IV, and (3) Investigator evaluation of performance by assessing whether the implant is palpably distinguishable from the tissue using a 5-point scale, where 1 means the Implant is very easy to distinguish from the tissue, 2 means the Implant is somewhat easy to distinguish from the tissue, 3 means the Implant is somewhat difficult to distinguish from the tissue, 4 means the Implant is very difficult to distinguish from the tissue, and 5 means the Implant is indistinguishable from the tissue.

Investigator assessment of whether the implant provides adequate projection, satisfaction with the device with regards to its use and clinical performance, and overall satisfaction relative to prior experience with the round textured breast implants are obtained along with any reasons for Investigator dissatisfaction.

Additional methods are employed to detect and monitor capsular contracture including HRUS, AT, and IT. Subject psychosocial well-being is measured using the BREAST-Q questionnaire. The BREAST-Q modules used in the study are designed and validated to assess the impact of breast surgery from the patient's perspective targeting two main concepts: (1) satisfaction with her breasts and (2) psychosocial well-being. See Pusic.

A separate statistical analysis plan provides specifications for all analyses. Descriptive statistics are presented for key outcome measures. Categorical variables are summarized with frequency and relative frequency. Continuous variables are summarized by number of subjects, mean, median, standard deviation, minimum, and maximum. Where appropriate, two-sided 95% confidence intervals for population mean, or population proportion, are provided as part of the descriptive summary. All implanted subjects are included in the full analysis (FA) population. The per-protocol (PP) population is defined as subjects who do not have any major protocol deviations throughout the study. Analysis of the primary outcome measure is performed on the PP population and repeated with the FA population as a sensitivity analysis. All other effectiveness analyses are performed on the PP population.

The primary effectiveness measure is the Investigator's overall satisfaction with the device for each subject 3 months post-implantation using a 5-point scale (see above). This variable is analyzed based on measurements as reported. No derivation of this variable is required. The primary effectiveness measure of the Investigator's overall satisfaction with the device at 3 months post-implantation is analyzed using descriptive statistics. Descriptive statistics include number of subjects, mean, standard deviation, median, minimum, maximum, and 95% confidence intervals as appropriate. In addition, frequency and relative frequency are presented at each score point. The primary measure analysis is based on the PP population and is repeated with the FA population as a sensitivity analysis.

Subject satisfaction with breasts 6 months post-implantation is measured using the BREAST-Q questionnaire. Summary scores are computed by summing the score of each response and transferring the sum to a 0 to 100 scale. Subject satisfaction is summarized by number of subjects, mean, standard deviation, median, minimum, maximum, change from baseline, and the 95% confidence intervals for the mean change. Summaries are performed based on the PP population.

All other effectiveness variables, including the primary and secondary variables at all other applicable time points, are summarized with descriptive statistics appropriate to the scale of measurement for each variable based on the PP population.

Investigator evaluation of whether the implant is palpably distinguishable from the tissue 6 months post-implantation is assessed on a 5-point scale and analyzed as reported. No further derivation of this variable is required. Determination of capsular contracture and its severity 2 years post-implantation are determined by Investigator rating of a subject's capsular contracture grade being either Grade III or Grade IV on the Baker Grade Classification, and are analyzed and reported without further derivation. Both the Investigator's evaluation of whether the implant is palpably distinguishable from the tissue at 6 months post-implantation and determination of capsular contracture 2 years post-implantation based on severity rating of Baker Grades III or IV are summarized descriptively. Summaries are performed based on the PP population.

The primary analysis is conducted once all subjects have completed the 6-month follow-up visit. Interim analyses are also conducted after all subjects have completed the 1-year and 2-year follow-up visits. Interim analyses may include all outcome measures available at the time of analysis, analyzed in accordance with the methods described above.

The textured, round breast implants are silicone-filled textured implants intended for breast augmentation and reconstruction surgeries. They are constructed with barrier shell technology resulting in a low diffusion silicone elastomer shell and are filled with a silicone gel. All profiles are single lumen design and consist of a shell, a patch, and silicone gel fill.

Standard B-mode (2D) HRUS are conducted using a standard HRUS machine (9 MHz). Settings are varied within common and acceptable clinical-use ranges, to measure the following 3 parameters: (1) distance from skin surface to inside surface of breast implant shell, (2) distance from skin surface to the innermost surface of either the gland or muscle tissue that immediately overlies the implant, and (3) dynamic assessment of the presence or absence of tissue integration with the implant. On each breast, these measures are determined at each of 4 sites: at 12 o'clock position, 3 o'clock position, 6 o'clock position, and 9 o'clock position, each at a distance from the nipple equal to half the nipple-inframammary fold distance. In total, 24 files are collected: 2 representative pictures and 1 video taken at each of the 4 sites on each breast.

AT is conducted by the Investigator according to the method described by Gahm et al., Mosahebi et al., and others. See Gahm et al., J Plast Reconstr Aesthet Surg. 2010; 63(2):332-338; Mosahebi et al., Plast Reconstr Surg. 2007; 119(3):796-803; Edsander-Nord et al., Scand J Plast Reconstr Surg Hand Surg 2004; 38: 204-2088; Gylbert, Scand J Plast Reconstr Surg Hand Surg 1989; 23: 223-2299; and, Moore, Plast Reconstr Surg 1979; 63: 9-12. A PLEXIGLASS® disc [poly(methylmethacrylate)/PMMA] of known mass (306 g) is placed at the central part of the breast with the subject lying supine. The disc is moistened with alcohol to show the area of contact with the breast more easily. The contact area (A) between the disc and the breast estimates the form of an ellipse and is calculated using the formula A=pi*a*b/4, where (a) is the largest diameter of the contact area in cm and (b) is the diameter perpendicular to (a) in cm. The weight of the Plexiglass disc provides a known force of 3.0N (F=ma=0.306 kg*9.80665 m/s²) that flattens the measured area (A). The intramammary pressure (P) can thus be estimated using the formula P=F/A in N/cm² (or kPa where 1N/cm²=10 kPa). The parameters measured are the (a) Maximum Contact Diameter recorded in mm for accuracy and (b) Perpendicular Contact Diameter recorded in mm for accuracy.

IT is conducted by the Investigator using a Shimpo FGE-20X Force Gauge (Electromatic Equipment Co., Inc., Cedarhurst, N.Y.) with 0.2N (10 g) resolution, a 100.0N (10 kg) capacity, and a 1.5 cm long 0.7 cm diameter round end tip. The parameter measured is the Force (in Newtons). With the subject in a supine position, on each breast the average of 3 measurements is determined at each of 5 sites: control site (upper pole of each breast over gland but not implant), and 4 implant sites (over the gland and implant) at 12 o'clock position, 3 o'clock position, 6 o'clock position, and 9 o'clock position, each at a distance from the nipple equal to half the nipple-inframammary fold distance.

A Baker Grade assessment of each breast is conducted by the Investigator, as described above.

Standard Breast Anatomical Measurements are conducted on each side by the Investigator, including (in cm): base width, nipple to inframammary fold, intermammary distance (distance between the breasts across the sternum), sternal notch to nipple, internipple distance, nipple to midline, and areolar diameter.

Breast photography is conducted by the Investigator, including the 8 following views: frontal (antero-posterior) with hands at rest by sides, frontal with hands at rest on waist, frontal with hands firmly pressing down on waist, lateral with hands at rest by sides (left and right side), diagonal (45°) with hands at rest by sides (left and right side), and a view of patient leaning forward towards camera with hands at rest on waist.

The study demonstrates that, in contrast to the body segregating the breast implant following implantation, providing a porous surface that promotes implant-tissue integration reduces capsule formation and/or capsular contracture rates.

In closing, it is to be understood that although aspects of the present specification have been described with reference to the various embodiments, one skilled in the art will readily appreciate that the specific examples disclosed are only illustrative of the principles of the subject matter disclosed in the present specification. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method for assessing occurrence and/or extent of capsular formation and/or occurrence and/or extent of capsular contraction in and around an implanted device in an individual, the method comprising obtaining one or more of assessments of an area in the vicinity of the implanted device, the one or more assessments comprising a first depth measurement from a skin surface of the individual, a second depth measurement from the skin surface of the individual, a tissue integration measurement of surrounding tissue with the implanted device, a tonometry measurement, a physical examination assessment, an anatomical assessment, a photographic assessment, or any combination thereof.
